(19) United States
(12) Patent Application Publication
(10) Pub. No.: US 2013/0236855 A1
(43) Pub. Date: Sep. 12, 2013

(54) PREMANUFACTURED CERAMIC DENTAL IMPLANT ABUTMENTS FOR MATING CERAMIC PROSTHETIC BUILD-UP CONSTRUCTION

(71) Applicant: JAMES R. GLIDEWELL DENTAL CERAMICS, INC., Newport Beach, CA (US)

(72) Inventors: Grant Bullis, Newport Beach, CA (US); Lieu Phan, Garden Grove, CA (US); Tao Nguyen, Anaheim, CA (US)

(21) Appl. No.: 13/788,649

(22) Filed: Mar. 7, 2013

Related U.S. Application Data
(60) Provisional application No. 61/608,534, filed on Mar. 8, 2012.

(51) Int. Cl. A61C 8/00; A61K 6/02

(52) U.S. Cl. CPC A61C 8/0012; A61C 8/0054; A61C 8/006; A61C 8/0072; A61K 6/024; A61C 8/0068
USPC 433/201.1

(57) ABSTRACT

A premade ceramic dental implant abutment includes a ceramic abutment having at the top a support region intended for receiving a mating prosthetic build-up construction with a surface feature to inhibit rotation of said construction, and at the bottom, an implant contact region with geometry to connect to the implant. The ceramic abutment has a passageway extending therethrough. The passageway may narrow to form a shoulder that is made entirely of ceramic. A head of a screw seats entirely on the shoulder. The ceramic abutment has a supragingival region for protruding beyond the gingiva and a subgingival end for extending into the gingiva. The ceramic abutment may be free of metal. Also, the premade ceramic abutment may be designed to be augmented by cementing additional ceramic material such as a ceramic prosthesis onto the ceramic abutment.

### RELATED APPLICATION

This application claims the benefit of and priority to U.S. Provisional Patent Application No. 61/608,534, filed on Mar. 8, 2012, the entirety of which application is incorporated herein by reference.

### FIELD

This invention generally relates to a ceramic abutment for use with a dental implant and adapted to support a prosthesis thereon. Specifically, the ceramic abutment is comprised of ceramic with no metal portion.

### BACKGROUND

Surgical techniques for support of dental prostheses by means of metallic endosseous artificial root fixtures are well documented in the dental literature. According to one prior art technique, a titanium, endosseous implant fixture is interfaced with a metallic abutment on which a superstructure, such as a prosthesis, is supported. The abutment has an internally shouldered access bore through which a screw is inserted to hold the implant and the abutment assembled. A number of problems and restrictions are presented, however, in the attachment and retention of the superstructures to such abutments. Specifically, the typical superstructures for use with dental implant abutments are made of dental ceramics. The use of a titanium abutment generally results in a dark, central shadow, particularly when exposed to bright light, which can compromise the esthetics of the restoration since it is visually different from a natural tooth.

Other problems exist with previous abutments that are designed to be used in their existing form or reduced to conform to a patient's unique anatomy. Such abutments require extensive modification if the implant is not placed at the correct angle and position to hold the predefined abutment contours in the correct orientation with the gingival tissues and adjacent anatomy. These abutments have predefined contours that assume an ideal placement of the implant which rarely is the case. Consequently, material must be ground away from the abutment to get correct occlusal and interproximal clearance, and there may be insufficient material in some regions to properly support the final prosthesis or gingival tissue.

### SUMMARY

A premade ceramic dental implant abutment and a screw, the ceramic portion having at the top a support region intended for receiving a mating prosthetic build-up construction with a surface feature to inhibit rotation of said construction, and at the bottom, an implant contact region with geometry to connect to the implant. The ceramic portion has a passageway extending through. According to one embodiment, the passageway narrows to form a shoulder that is made entirely of ceramic. The screw comprises a head and a threaded section. The head of the screw seats entirely on the shoulder. The ceramic abutment has a supragingival region for protruding beyond the gingiva and a subgingival end for extending into the gingiva.

The ceramic abutment of the present disclosure is designed to be augmented by the cementation of additional ceramic material such as a ceramic prosthesis onto an exterior surface shoulder of the ceramic abutment. This construction allows a laboratory greater latitude to adapt the abutment geometry to the specific anatomy of the patient and the implant placement.

In an embodiment, a premanufactured ceramic dental implant abutment includes: a top portion comprising a support region for receiving a mating prosthetic, the top portion having a surface feature to inhibit rotation of the mating prosthetic, a bottom portion comprising an implant contact region configured to connect with a dental implant, and a passageway extending through at least a portion of the top portion and the bottom portion, the passageway configured to receive an abutment screw to secure the abutment to the dental implant. The top portion and the bottom portion comprise a ceramic and are free of metal. The abutment screw may include a head and a threaded section, and the passageway narrows to form a shoulder having a surface that provides a seat for the head of the screw.

The support region may be a generally cylindrical section. The generally cylindrical section may be configured to engage the mating prosthesis. The surface feature may be a flat surface portion on the generally cylindrical section. The flat surface portion may be rectangular in shape.

The ceramic of the top portion and the bottom portion may comprise yttria-stabilized zirconium oxide. The prosthetic may include a ceramic coping.

In an embodiment, a premanufactured ceramic dental implant abutment includes: a top portion comprising a generally cylindrical section for receiving a mating ceramic prosthetic, the top portion having a surface feature to inhibit rotation of the mating ceramic prosthetic, a bottom portion comprising an implant contact region configured to connect with a dental implant, and a passageway extending through at least a portion of the top portion and the bottom portion. The passageway is configured to receive an abutment screw to secure the abutment to the dental implant, and the top portion and the bottom portion comprise a ceramic and are free of metal.

The surface feature may be a flat surface portion on the generally cylindrical section. The flat surface portion may be rectangular in shape. The generally cylindrical section may be tapered to aid in securing the mating ceramic prosthetic to the top portion. The generally cylindrical section may include at least one groove to secure a set screw to secure the mating ceramic prosthetic.

The ceramic of the top portion and the bottom portion may comprise yttria-stabilized zirconium oxide. Also, the mating ceramic prosthetic may include a ceramic coping.

In an embodiment, a premanufactured dental implant abutment system includes: a premanufactured ceramic abutment that is free of metal and an abutment screw. The ceramic abutment includes: a top portion comprising a support region for receiving a mating ceramic prosthetic, the top portion having a surface feature to inhibit rotation of the mating ceramic prosthetic, a bottom portion comprising an implant contact region configured to connect with a dental implant, and a passageway extending through the abutment. The abutment screw is configured for insertion in the passageway and includes a head and a threaded section. The passageway forms a shoulder that provides a seat for the head of the abutment screw.

The support region may be a generally cylindrical section. The surface feature may be a flat surface portion on the generally cylindrical section. The generally cylindrical section may include at least one groove to secure a set screw to secure the mating ceramic prosthetic to the ceramic abutment. The ceramic abutment may comprise yttria-stabilized zirconium oxide.

### BRIEF DESCRIPTION OF THE DRAWINGS

The aforementioned objects and advantages, as well as additional objects and advantages thereof, will be more fully understood hereinafter as a result of a detailed description of various embodiments when taken in conjunction with the following drawings in which:

FIG. 1 is an exploded perspective view of a premade ceramic dental implant abutment and a screw having at the top a support region intended for receiving a mating prosthetic build-up construction with a surface feature to inhibit rotation of said construction, and at the bottom, an implant contact region with geometry to connect to the implant;

FIG. 2 is a side cross-sectional view of a premade ceramic dental implant abutment of FIG. 1;

FIG. 3 is a cross-sectional view of a premade ceramic dental implant abutment and an abutment screw according to one embodiment of the present disclosure;

FIG. 4 is a top view of the premade ceramic dental implant abutment of FIG. 1;

FIG. 5 is a bottom view of the ceramic dental implant abutment of FIG. 1; and

FIG. 6 is a side cross-sectional view of the ceramic dental implant abutment of FIG. 1 with a ceramic coping.

### DETAILED DESCRIPTION

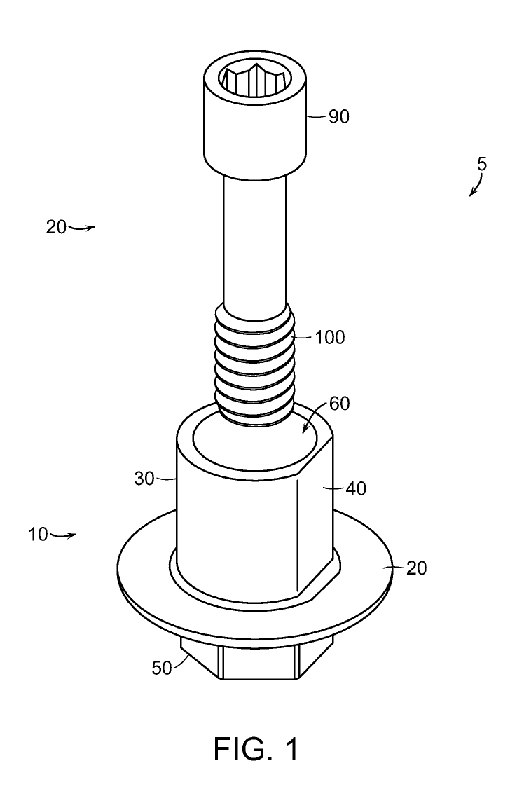

Referring now to the drawings in detail, FIG. 1 shows an exploded perspective view of a premade ceramic dental implant abutment 10. The premade ceramic dental implant abutment 10 is designed to support a mating ceramic coping thereon. The ceramic coping and the premade ceramic dental implant abutment 10 reproduce the shape and gingival contours of the tooth being replaced.

FIG. 1 shows a premanufactured dental implant abutment system 5 including a premade ceramic dental implant abutment 10 and a screw 20 having at the top a support region 30 intended for receiving a mating prosthetic build-up construction with a surface feature 40 to inhibit rotation of said construction, and at the bottom, an implant contact region 50 with geometry to connect to the implant.

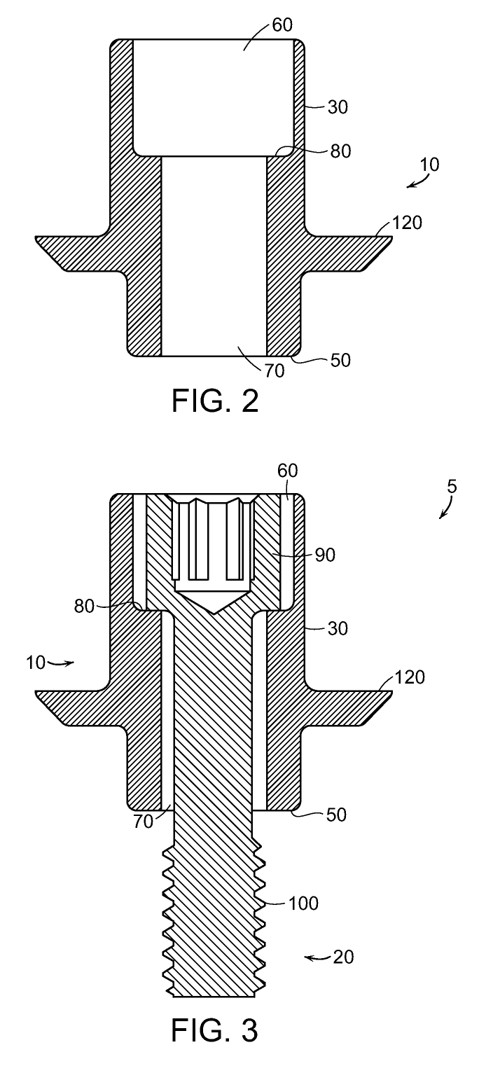

FIG. 2 is a side cross-sectional view of a premade ceramic dental implant abutment 10 of FIG. 1.

FIG. 3 is a cross-sectional view of a premade ceramic dental implant abutment 10 and an abutment screw 20 according to one embodiment of the present disclosure.

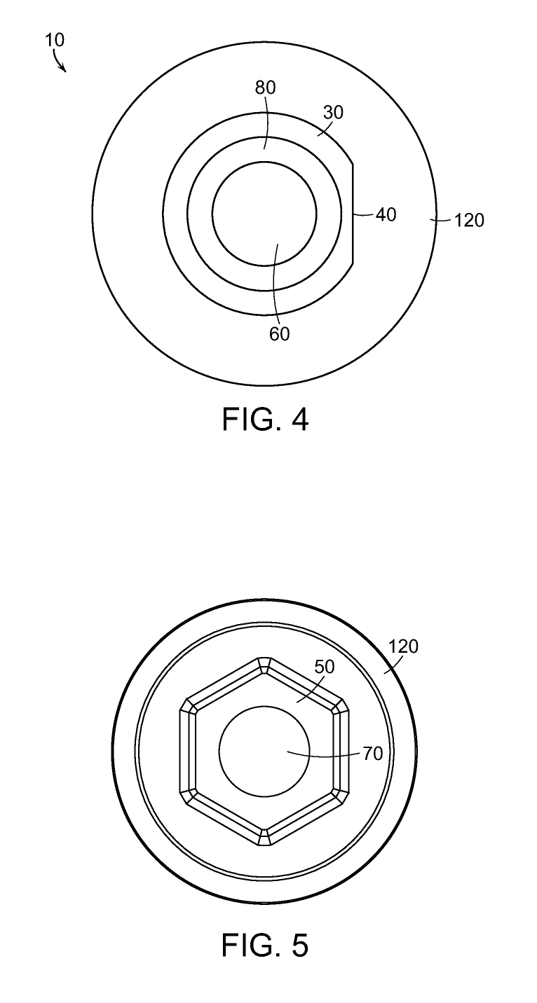

FIG. 4 is a top view of the premade ceramic dental implant abutment 10 of FIG. 1.

FIG. 5 is a bottom view of the ceramic dental implant abutment 10 of FIG. 1.

To provide access for a screw 20 that engages threads of an internal bore in the dental implant, the premade ceramic dental implant abutment 10 has a passageway therethrough. According to one embodiment, a passageway 70 narrows to form a shoulder 80 that is made entirely of ceramic. The narrow portion of the passageway 70 may be referred to as a central opening 60. The central opening 60 extends upwardly into the passageway 70 of the ceramic abutment 10. In FIG. 3, a screw 20 is shown extending through the central opening 60. The screw 20 includes a head 90 and a lower section having external threads 100 thereon.

The screw 20 is threaded into an internally threaded bore of the dental implant The screw 20 secures the premade ceramic dental implant abutment 10 and the dental implant. The dental implant also has threads for engaging the bone of the dental arches (including the maxillary and mandible). The dental implant is located below a gingival surface and contacts a dental arch (not shown). The premade ceramic dental implant abutment 10 includes a supragingival section that protrudes beyond the gingiva, and a subgingival section that extends into the gingiva. The supragingival section may include a support region 30 that is a generally cylindrical section.

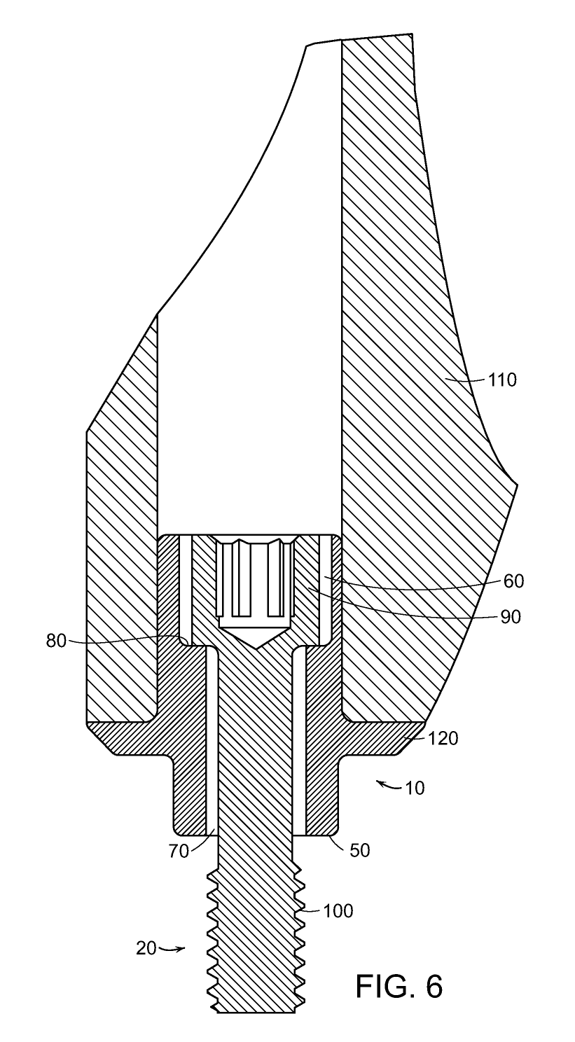

The generally cylindrical section of the premade ceramic dental implant abutment 10 is adapted to engage a prosthesis (FIG. 6).

FIG. 6 is a side cross-sectional view of the ceramic dental implant abutment 10 of FIG. 1 with a ceramic coping 110.

The generally cylindrical section may be formed into other shapes. The generally cylindrical section has a surface feature 40 such as a generally flat surface portion. The flat surface portion may be generally rectangular in shape (see FIG. 1), but may be formed in other shapes, such as, for example, a triangular shape, or a round shape. The flat surface portion or other surface feature 40 inhibits rotation of a prosthesis (FIG. 6) relative to the premade ceramic dental implant abutment 10. Specifically, rotation is inhibited or prevented when an adhesive is added to secure a placed prosthesis to the premade ceramic dental implant abutment 10. The adhesive is placed in a space formed between an interior surface of a prosthesis and the surface feature 40 or flat surface portion.

According to one process, the abutment 10 is altered by cementing a coping which reproduces the shape and gingival contours of the tooth being replaced. The abutment 10 and coping may be stained to match the color of the adjacent teeth.

Additionally, the generally cylindrical section may be tapered slightly to further aid in installing and securing a prosthesis to the premade ceramic dental implant abutment 10.

Optionally, the generally cylindrical section may have a groove (not shown) or other means for securing a set screw used to hold a prosthesis in place. The exterior surface of the generally cylindrical section has an exterior surface shoulder 120. The exterior surface shoulder 120 may protrude outwardly from the generally cylindrical section. The exterior surface shoulder 120 is connected to a generally cylindrical exterior surface that extends generally in a downward direction towards a dental implant. The generally cylindrical exterior surface is connected to a tapered surface that extends inwardly and downwardly towards a dental implant.

The premade ceramic abutment 10 of the present disclosure is designed to be augmented by the cementation of additional ceramic material such as a ceramic prosthesis onto an exterior surface shoulder 120 of the ceramic abutment. For example, the ceramic abutment 10 may be designed to have a prosthetic such as a ceramic coping crown cemented onto the ceramic abutment 10 to create the final restoration.

Referring specifically to FIG. 1, the premade ceramic abutment 10 is hollow with the passageway 70 therethrough. At an occlusal end of the premade ceramic abutment 10, there is an open end which allows a screw 20 to enter into the premade ceramic abutment 10. The passageway 70 narrows to form a shoulder 80 that provides a seat for a screw 20, such as the head of the screw 20 as shown in FIG. 2. Specifically, the passageway 70 has a generally horizontal surface defining the shoulder 80. The shoulder 80 of FIG. 2 is made entirely of ceramic.

The shoulder 80 length of the premade ceramic abutment 10 may be varied to accommodate differing patient's anatomies. For example, if a patient has a greater gingiva height, the length may be increased. The length is generally from about 0.5 mm to about 4.0 mm. The length may, however, be less than 0.5 mm and greater than 4.0 mm. The length varies so as to be aesthetically pleasing by remaining below the gingival surface. The width of the premade ceramic abutment 10 may also vary to accommodate differing patient's anatomies.

A premade ceramic abutment 10 made from yttria-stabilized zirconium oxide generally has a greater fracture toughness and flexural strength than a ceramic portion of aluminum oxide. It is desirable to have a higher fracture toughness and flexural strength because the ceramic portion is stronger, tougher, and more durable.

In an embodiment, the premade ceramic abutment 10 is comprised entirely of ceramic with no metal portion.

While the present invention has been described with reference to one or more particular embodiments, those skilled in the art will recognize that many changes may be made thereto without departing from the spirit and scope of the present invention. Each of these embodiments and variations thereof is contemplated as falling within the spirit and scope of the invention.

We claim:

1. A premanufactured ceramic dental implant abutment comprising:
a top portion comprising a support region for receiving a mating prosthetic, the top portion having a surface feature to inhibit rotation of the mating prosthetic,
a bottom portion comprising an implant contact region configured to connect with a dental implant, and
a passageway extending through at least a portion of the top portion and the bottom portion, the passageway configured to receive an abutment screw to secure the abutment to the dental implant,
wherein the top portion and the bottom portion comprise a ceramic and are free of metal.

2. The abutment of claim 1, wherein the abutment screw includes a head and a threaded section, and the passageway narrows to form a shoulder having a surface that provides a seat for the head of the screw.

3. The abutment of claim 1, wherein the support region is a generally cylindrical section.

4. The abutment of claim 3, wherein the generally cylindrical section is configured to engage the mating prosthesis.

5. The abutment of claim 4, wherein the surface feature is a flat surface portion on the generally cylindrical section.

6. The abutment of claim 5, wherein the flat surface portion is rectangular in shape.

7. The abutment of claim 1, wherein the ceramic of the top portion and the bottom portion comprises yttria-stabilized zirconium oxide.

8. The abutment of claim 1, wherein the prosthetic includes a ceramic coping.

9. A premanufactured ceramic dental implant abutment comprising:
a top portion comprising a generally cylindrical section for receiving a mating ceramic prosthetic, the top portion having a surface feature to inhibit rotation of the mating ceramic prosthetic,
a bottom portion comprising an implant contact region configured to connect with a dental implant, and
a passageway extending through at least a portion of the top portion and the bottom portion, the passageway configured to receive an abutment screw to secure the abutment to the dental implant, wherein the top portion and the bottom portion comprise a ceramic and are free of metal.

10. The abutment of claim 9, wherein the surface feature is a flat surface portion on the generally cylindrical section.

11. The abutment of claim 10, wherein the flat surface portion is rectangular in shape.

12. The abutment of claim 9, wherein the generally cylindrical section is tapered to aid in securing the mating ceramic prosthetic to the top portion.

13. The abutment of claim 9, wherein the generally cylindrical section includes at least one groove to secure a set screw to secure the mating ceramic prosthetic.

14. The abutment of claim 9, wherein the ceramic of the top portion and the bottom portion comprises yttria-stabilized zirconium oxide.

15. The abutment of claim 9, wherein the mating ceramic prosthetic includes a ceramic coping.

16. A premanufactured dental implant abutment system comprising:
a premanufactured ceramic abutment comprising:
a top portion comprising a support region for receiving a mating ceramic prosthetic, the top portion having a surface feature to inhibit rotation of the mating ceramic prosthetic,
a bottom portion comprising an implant contact region configured to connect with a dental implant, and
a passageway extending through the abutment; and
an abutment screw for insertion in the passageway, the abutment screw comprising a head and a threaded section,
wherein the passageway forms a shoulder that provides a seat for the head of the abutment screw, and
wherein the ceramic abutment is free of metal.

17. The abutment system of claim 16, wherein the support region is a generally cylindrical section.

18. The abutment system of claim 16, wherein the surface feature is a flat surface portion on the generally cylindrical section.

19. The abutment system of claim 16, wherein the generally cylindrical section includes at least one groove to secure a set screw to secure the mating ceramic prosthetic to the ceramic abutment.

20. The abutment system of claim 16, wherein the ceramic abutment comprises yttria-stabilized zirconium oxide.
